(19) United States
(12) Patent Application Publication
(10) Pub. No.: US 2008/0002806 A1
(43) Pub. Date: Jan. 3, 2008

(54) X-RAY CT APPARATUS AND X-RAY CT IMAGING METHOD

(76) Inventors: Akihiko Nishide, Tokyo (JP); Makoto Gohno, Tokyo (JP); Masatake Nukui, Tokyo (JP); Akira Hagiwara, Tokyo (JP); Kotoko Morikawa, Tokyo (JP)

Correspondence Address: Patrick W. Rasche;Armstrong Teasdale LLP, Suite 2600, One Metropolitan Square, St. Louis, MO, 63102, US

(21) Appl. No.: 11/769,424

(22) Filed: Jun. 27, 2007

(30) Foreign Application Priority Data
Jun. 29, 2006 (JP) 2006-178873

(51) Int. Cl. H05G 1/60; A61B 6/00; G01N 23/00; G21K 1/12

(52) U.S. Cl. 378/4; 378/15; 378/901

(57) ABSTRACT

Improvement of the picture quality of tomograms in an X-ray CT apparatus using a multi-row x-ray detector is to be realized. When conventional scanning (axial scanning) or cine-scanning is to be performed in consecutive different scanning positions in the z-axis direction, the width of the X-ray beam at scanning positions at both ends is kept at exactly or approximately D/2 relative to the multi-row x-ray detector. Alternatively, the interval between one scanning position and another is kept at not more than D. Unevenness of picture quality dependent on positions on the z-axis on a reconstructed plane can be improved.

### CROSS REFERENCE TO RELATED APPLICATIONS

This application claims the benefit of Japanese Patent Application No. 2006-178873 filed Jun. 29, 2006.

### BACKGROUND OF THE INVENTION

The present invention relates to an X-ray CT (Computed Tomography) apparatus and an X-ray CT imaging method, and more particularly to an X-ray CT apparatus and an X-ray CT imaging method which enable, when conventional scanning (axial scanning) or cine-scanning by an X-ray CT apparatus having an X-ray area detector of matrix structure, typically a multi-row X-ray detector or a flat panel, is to be performed in consecutive different scanning positions in the body axis direction (z-axis direction) of a subject, picture quality unevenness dependent on the position of the reconstructed plane to be improved and any wastefully irradiated area to be reduced.

Techniques by which conventional scanning by an X-ray CT apparatus having a multi-row X-ray detector is performed in consecutive different scanning positions in the z-axis direction are already known (see JP-A No. 250794/2003 for instance).

On the other hand, in order to prevent irradiation of an area farther ahead in the direction of linear transfer than the linear transfer area in which projection data are to be acquired when helical scanning is to be performed, there are known X-ray CT apparatuses which restrict with a collimator forward in the direction of linear transfer the end face position of the X-ray beam in an area forward in the direction of linear transfer at the time of starting irradiation with X-rays, and which restrict with a collimator backward in the direction of linear transfer the end face position of the X-ray beam in an area backward in the direction of linear transfer at the time of ending irradiation with X-rays (see JP-A No. 320609/2002 for instance).

FIG. 28 shows a first prior art case in which conventional scanning or cine-scanning by an X-ray CT apparatus having a multi-row X-ray detector 24 is performed in consecutive different scanning positions in the z-axis direction.

In this first prior art case, conventional scanning or cine-scanning is performed in different scanning positions in the z-axis direction, z1, z3 (=z1+D), z5 (=z3+D) and z7 (=z5+D), and tomograms on reconstruction planes P0 through P8 or tomograms in random positions between P0 and P8 are subjected to image reconstruction on the basis of projection data that have been acquired. In these equations, D is the width of a multi-row X-ray detector 24 in the z-axis direction on the rotation center axis IC of an X-ray tube 21 and the multi-row X-ray detector 24 as the multi-row X-ray detector 24 is viewed from the focus of the X-ray tube 21, and is about ½ of the width of the actual multi-row X-ray detector 24 in the z-axis direction.

FIG. 29 shows conventional scanning or cine-scanning in the scanning position z1. FIG. 30 shows conventional scanning or cine-scanning in the scanning position z3.

Projected data for subjecting pixels on the rotation axis of the tomogram on the reconstruction plane P0 to image reconstruction can only be obtained by conventional scanning or cine-scanning in the scan position z1 shown in FIG. 29 because the reconstruction plane P0 is positioned at an end. Moreover, projection data on the pixel g in reconstruction plane P0 shown in FIG. 29, for instance, can be obtained at the view angle shown in FIG. 29(b), but not at the view angle shown in FIG. 29(a). Moreover, the X-ray beam CB is greatly inclined relative to the reconstruction plane P0. This results in a problem that the picture quality of the tomogram on the reconstruction plane P0 is degraded by artifact occurrence. Similarly, there also is a problem that the picture quality of the tomogram on the reconstruction plane P8 at the other end is also degraded. Furthermore, there is still another problem that wastefully irradiated areas emerge outside the reconstruction planes P0 and P8 at the two ends.

Then, though projection data for subjecting the tomogram on the reconstruction plane P1 can only be obtained by conventional scanning or cine-scanning in the scan position z1 shown in FIG. 29, they can be obtained on any pixel in every view angle. Furthermore, the X-ray beam CB is not inclined relative to the reconstruction plane P1. As a result, the picture quality of the tomogram on the reconstruction plane P1 is sufficiently high.

Next, projection data for subjecting the tomogram on the reconstruction plane P2 can be obtained by conventional scanning or cine-scanning in the scan position z1 shown in FIG. 29 and conventional scanning or cine-scanning in the scan position z3 shown in FIG. 30. However, for instance projection data on the pixel g on the reconstruction plane P2 shown in FIG. 29 and FIG. 30 can be obtained at the view angle shown in FIG. 29(b) and FIG. 30(b), but not at the view angle shown in FIG. 29(a) and FIG. 30(a). Furthermore, the X-ray beam CB is greatly inclined relative to the reconstruction plane P2. As a result, there is a problem that the picture quality of the tomogram on the reconstruction plane P2, though better than that of the tomogram on the reconstruction plane P0, is inferior to that of the tomogram on the reconstruction plane P1.

FIG. 31 shows a second prior art case in which conventional scanning or cine-scanning by an X-ray CT apparatus having a multi-row X-ray detector is performed in consecutive different scanning positions in the z-axis direction.

In this second prior art case, conventional scanning or cine-scanning is performed in different scanning positions in the z-axis direction, z0, z2, z4, z6 and z8, and tomograms on the reconstruction planes P0 through P8 are subjected to image reconstruction on the basis of projection data that have been acquired.

In this case, the picture quality of the tomograms on the reconstruction planes P0, P2 and P8 is sufficiently high. However, there is a problem that the picture quality of the tomogram on the reconstruction plane P1 is inferior to that of the tomograms on the reconstruction planes P0, P2 and P8.

Therefore, an object of the present invention is to improve unevenness of picture quality dependent on the position of the reconstructed plane when conventional scanning or cine-scanning by an X-ray CT apparatus having a multi-row X-ray detector is performed in consecutive different scanning positions in the z-axis direction.

### SUMMARY OF THE INVENTION

According to its first aspect, the invention provides an X-ray CT apparatus characterized in that it is equipped with a projection data acquisition device for acquiring, while rotating an X-ray generating device and a multi-row X-ray detector opposite the X-ray generating device within an xy plane around a rotation center axis positioned between the X-ray generating device and the multi-row X-ray detector, projection data of a subject positioned in-between; a collimator for controling the opening width of an X-ray beam irradiating the X-ray area detector in a direction perpendicular to the xy plane; a scanning table for transferring the subject in the z-axis direction; an image reconstruction device for image-reconstructing tomograms on the basis of the projection data that have been acquired; an image display for displaying the tomograms having undergone the image reconstruction; a scanning condition setting device for setting various scanning conditions for acquiring the projection data; and a controller for controlling, when performing conventional scanning (axial scanning) or cine-scanning in consecutive different scanning positions in the z-axis direction, at both scan positions, the collimator as to make the width of the X-ray beam D/2 or approximately D/2 relative to a multi-row X-ray detector width D on the rotation center axis or to make the expanding angle of the X-ray beam θ/2 or approximately θ/2 relative to a detector angle θ, and controlling the scanning table as to keep the interval between one scanning position and another scanning position at not more than D.

The X-ray CT apparatus according to the first aspect, once the reconstruction plane is set within the range of the first scanning position to the final scanning position, can obtain projection data in every view angle for any pixel on reconstruction planes at both ends, and reduces the inclination of the X-ray beam relative to the reconstruction plane. As a result, picture quality of tomograms becomes sufficiently even on the reconstruction planes at both ends. Also, since the interval between one scanning position and another scanning position is kept at not more than D, the inclination of the X-ray beam and its fluctuations can be reduced on a reconstruction plane positioned between one scanning position and another scanning position, thereby making it possible to improve the picture quality of tomograms. Therefore, unevenness of picture quality dependent on the position of the reconstructed plane can be improved. Furthermore, since the width of the X-ray beam is narrowed in scanning positions at both ends, any wastefully irradiated area can be reduced.

According to its second aspect, the invention provides an X-ray CT apparatus characterized in that it is equipped with a projection data acquisition device for acquiring, while rotating an X-ray generating device and a multi-row X-ray detector opposite the X-ray generating device within an xy plane around a rotation center axis positioned between the X-ray generating device and the multi-row X-ray detector, projection data of a subject positioned in-between; a collimator for controling the opening width of an X-ray beam irradiating the X-ray area detector in a direction perpendicular to the xy plane; a scanning table for transferring the subject in the z-axis direction; an image reconstruction device for image-reconstructing tomograms on the basis of the projection data that have been acquired; an image display for displaying the tomograms having undergone the image reconstruction; a scanning condition setting device for setting various scanning conditions for acquiring the projection data; and a controller for controlling, when performing conventional scanning (axial scanning) or cine-scanning in consecutive different scanning positions in the z-axis direction, at both scan positions, the collimator as to make the width of the X-ray beam D/2 or approximately D/2 relative to a multi-row X-ray detector width D on the rotation center axis or to make the expanding angle of the X-ray beam θ/2 or approximately θ/2 relative to a detector angle θ.

The X-ray CT apparatus according to the second aspect, once the reconstruction plane is set within the range of the first scanning position to the final scanning position, can obtain projection data in every view angle for any pixel on reconstruction planes at both ends, and reduces the inclination of the X-ray beam relative to the reconstruction plane. Furthermore, since the width of the X-ray beam is narrowed in scanning positions at both ends, any wastefully irradiated area can be reduced.

According to its third aspect, the invention provides an X-ray CT apparatus characterized in that it is equipped with a projection data acquisition device for acquiring, while rotating an X-ray generating device and a multi-row X-ray detector opposite the X-ray generating device within an xy plane around a rotation center axis positioned between the X-ray generating device and the multi-row X-ray detector, projection data of a subject positioned in-between; a collimator for controling the opening width of an X-ray beam irradiating the X-ray area detector in a direction perpendicular to the xy plane; a scanning table for transferring the subject in the z-axis direction; an image reconstruction device for image-reconstructing tomograms on the basis of the projection data that have been acquired; an image display for displaying the tomograms having undergone the image reconstruction; a scanning condition setting device for setting various scanning conditions for acquiring the projection data; and a controller for controlling, when performing conventional scanning (axial scanning) or cine-scanning in consecutive different scanning positions in the z-axis direction, the scanning table as to keep the interval between one scanning position and another scanning position at not more than D relative to a multi-row X-ray detector width D on the rotation center axis.

The X-ray CT apparatus according to the third aspect, since it keeps the interval between one scanning position and another scanning position at not more than D, it can reduce the inclination of the X-ray beam and its fluctuations on a reconstruction plane positioned between one scanning position and another scanning position, thereby making it possible to improve the picture quality of tomograms. Therefore, unevenness of picture quality dependent on the position of the reconstructed plane can be improved.

According to its fourth aspect, the invention provides the X-ray CT apparatus according to any of the first through third aspects, characterized in that it is equipped with a projection data synthesizing device for synthesizing projection data for image reconstruction by subjecting projection data which have been acquired in different scanning positions and match the X-ray beam passing the same pixel on the reconstruction plane to interpolation or weighted addition.

The X-ray CT apparatus according to the fourth aspect, as it synthesizes projection data acquired in different scanning positions at the projection data stage, has an advantage of requiring only one step of image reconstruction computing.

According to its fifth aspect, the invention provides the X-ray CT apparatus according to any of the first through third aspects, characterized in that it is equipped with a projection data synthesizing device for synthesizing projection data for image reconstruction by subjecting projection data which have been acquired in different scanning positions and match the X-ray beam passing the same pixel or the vicinities of the pixel on the reconstruction plane to interpolation or weighted addition.

The X-ray CT apparatus according to the fifth aspect, as it synthesizes projection data acquired in different scanning positions at the projection data stage, has an advantage of requiring only one step of image reconstruction computing. Also, as it synthesizes not only projection data passing the same pixel on the reconstruction plane but also projection data passing the vicinities of the pixel, picture quality can be improved.

According to its sixth aspect, the invention provides the X-ray CT apparatus according to the fifth aspect, characterized in that the vicinities are a prescribed range in the z-axis direction centering on the pixel.

The X-ray CT apparatus according to the sixth aspect can subject a tomogram of a desired width in the z-axis direction to image reconstruction.

According to its seventh aspect, the invention provides the X-ray CT apparatus according to any of the fourth through sixth aspects, characterized in that the interpolation coefficient for the interpolation or the weighted addition coefficient for the weighted addition is determined on the basis of the geometrical positions and directions of the X-ray beams passing the pixels matching the sets of projection data to be subjected to interpolation or weighted addition.

The X-ray CT apparatus according to the seventh aspect, as it controls the interpolation coefficient or the weighted addition coefficient according to the geometrical positions and directions of the X-ray beams, can improve picture quality by reducing artifacts.

According to its eighth aspect, the invention provides the X-ray CT apparatus according to any of the first through third aspects, characterized in that the image reconstruction device is equipped with a tomogram synthesizing device for synthesizing a new tomogram by subjecting tomograms from projection data acquired in the same scanning position to image reconstruction and subjecting tomograms having undergone image reconstruction from projection data on the same reconstruction plane in different scanning positions to interpolation or weighted addition on a pixel-by-pixel basis.

The X-ray CT apparatus according to the eighth aspect, as it synthesizes tomograms on the basis of projection data acquired in different scanning positions and synthesizes them at the projection data stage, has an advantage of obtaining tomograms of a plurality of types.

According to its ninth aspect, the invention provides the X-ray CT apparatus according to the eighth aspect, characterized in that the image reconstruction device is equipped with the tomogram synthesizing device which synthesizes a new tomogram by subjecting tomograms on one or more reconstruction planes from projection data acquired in the same scanning position to image reconstruction and subjecting tomograms having undergone image reconstruction from projection data on a reconstruction plane contained in a prescribed range in the z-axis direction in the same scanning position and in different scanning positions to interpolation or weighted addition on a pixel-by-pixel basis.

The X-ray CT apparatus according to the ninth aspect, as it synthesizes tomograms on the basis of projection data acquired in different scanning positions and synthesizes them at the projection data stage, has an advantage of obtaining tomograms of a plurality of types. Also, as it synthesizes not only tomograms on the same reconstruction plane but also tomograms on a reconstruction plane contained in a prescribed range in the z direction, can subject tomograms of a prescribed width in the z-axis direction to image reconstruction.

According to its tenth aspect, the invention provides the X-ray CT apparatus according to the eighth aspect or the ninth aspect, characterized in that the interpolation coefficient for the interpolation or the weighted addition coefficient for the weighted addition is determined on the basis of the geometrical positions and directions of the X-ray beams passing the pixels of the tomograms to be subjected to interpolation or weighted addition on the pixel-by-pixel basis.

The X-ray CT apparatus according to the tenth aspect, as it controls the interpolation coefficient or the weighted addition coefficient according to the geometrical positions and directions of the X-ray beams, can improve picture quality by reducing artifacts.

According to its eleventh aspect, the invention provides an X-ray CT imaging method for acquiring projection data of a subject positioned between an X-ray generating device and a multi-row X-ray detector opposite the X-ray generating device while rotating the X-ray generating device and the multi-row X-ray detector within an xy plane around a rotation center axis positioned between them, wherein, when performing conventional scanning (axial scanning) or cine-scanning in consecutive different scanning positions in the z-axis direction orthogonal to the xy plane, at both scan positions, the width of the X-ray beam in the z-axis direction is made D/2 or approximately D/2 relative to a multi-row X-ray detector width D on the rotation center axis or the expanding angle of the X-ray beam in the z-axis direction is made θ/2 or approximately θ/2 relative to a detector angle θ, and the interval between one scanning position and another scanning position is kept at not more than D.

By the X-ray CT imaging method according to the eleventh aspect, once the reconstruction plane is set within the range of the first scanning position to the final scanning position, projection data in every view angle for any pixel on reconstruction planes at both ends can be obtained, and the inclination of the X-ray beam relative to the reconstruction plane is reduced. As a result, picture quality of tomograms becomes sufficiently even on the reconstruction planes at both ends. Also, since the interval between one scanning position and another scanning position is kept at not more than D, the inclination of the X-ray beam and its fluctuations can be reduced on a reconstruction plane positioned between one scanning position and another scanning position, thereby making it possible to improve the picture quality of tomograms. Therefore, unevenness of picture quality dependent on the position of the reconstructed plane can be improved. Furthermore, since the width of the X-ray beam is narrowed in scanning positions at both ends, any wastefully irradiated area can be reduced.

According to its twelfth aspect, the invention provides an X-ray CT imaging method for acquiring projection data of a subject positioned between an X-ray generating device and a multi-row X-ray detector opposite the X-ray generating device while rotating the X-ray generating device and the multi-row X-ray detector within an xy plane around a rotation center axis positioned between them, wherein, when performing conventional scanning (axial scanning) or cine-scanning in consecutive different scanning positions in the z-axis direction orthogonal to the xy plane, at both scan positions, the width of the X-ray beam in the z-axis direction is made D/2 or approximately D/2 relative to a multi-row X-ray detector width D on the rotation center axis or the expanding angle of the X-ray beam in the z-axis direction is made θ/2 or approximately θ/2 relative to a detector angle θ.

By the X-ray CT imaging method according to the twelfth aspect, once the reconstruction plane is set within the range of the first scanning position to the final scanning position, projection data in every view angle for any pixel on reconstruction planes at both ends can be obtained, and the inclination of the X-ray beam relative to the reconstruction plane is reduced. As a result, picture quality of tomograms becomes sufficiently even on the reconstruction planes at both ends. Also, since the width of the X-ray beam is narrowed in scanning positions at both ends, any wastefully irradiated area can be reduced.

According to its thirteenth aspect, the invention provides an X-ray CT imaging method for acquiring projection data of a subject positioned between an X-ray generating device and a multi-row X-ray detector opposite the X-ray generating device while rotating the X-ray generating device and the multi-row X-ray detector within an xy plane around a rotation center axis positioned between them, wherein, when performing conventional scanning (axial scanning) or cine-scanning in consecutive different scanning positions in the z-axis direction orthogonal to the xy plane, the interval between one scanning position and another scanning position is kept at not more than D.

By the X-ray CT imaging method according to the thirteenth aspect, since the interval between one scanning position and another scanning position is kept at not more than D, the inclination of the X-ray beam and its fluctuations on a reconstruction plane positioned between one scanning position and another scanning position can be reduced, thereby making it possible to improve the picture quality of tomograms. Therefore, unevenness of picture quality dependent on the position of the reconstructed plane can be improved.

According to its fourteenth aspect, the invention provides the X-ray CT imaging method according to any of the eleventh th aspect through the thirteenth aspect, characterized in that tomograms are subjected to image reconstruction on the basis of projection data obtained by subjecting projection data which have been acquired in different scanning positions and match the X-ray beam passing the same pixel on the reconstruction plane to interpolation or weighted addition.

The X-ray CT imaging method according to the fourteenth aspect, since it synthesizes projection data acquired in different scanning positions at the projection data stage, provides an advantage of requiring only one step of image reconstruction computing.

According to its fifteenth aspect, the invention provides the X-ray CT imaging method according to any of the eleventh aspect through the thirteenth aspect, characterized in that projection data for image reconstruction are synthesized by subjecting projection data which have been acquired in different scanning positions and match the X-ray beam passing the same pixel or the vicinities of the pixel on the reconstruction plane to interpolation or weighted addition.

The X-ray CT imaging method according to the fifteenth aspect, since it synthesizes projection data acquired in different scanning positions at the projection data stage, provides an advantage of requiring only one step of image reconstruction computing. Also, as it synthesizes not only projection data passing the same pixel on the reconstruction plane but also projection data passing the vicinities of the pixel, picture quality can be improved.

According to its sixteenth aspect, the invention provides the X-ray CT imaging method according to the fifteenth aspect, characterized in that the vicinities are a prescribed range in the z-axis direction centering on the pixel.

By the X-ray CT imaging method according to the sixteenth aspect, a tomogram of a desired width in the z-axis direction can be subjected to image reconstruction.

According to its seventeenth aspect, the invention provides the X-ray CT imaging method according to any of the fourteenth aspect through the sixteenth aspect, characterized in that the interpolation coefficient for the interpolation or the weighted addition coefficient for the weighted addition is determined on the basis of the geometrical positions and directions of the X-ray beams passing the pixels matching the sets of projection data to be subjected to interpolation or weighted addition. By the X-ray CT imaging method according to the seventeenth aspect, as the interpolation coefficient or the weighted addition coefficient is controlled according to the geometrical positions and directions of the X-ray beams, picture quality can be improved by reducing artifacts.

According to its eighteenth aspect, the invention provides the X-ray CT imaging method according to any of the eleventh aspect through the thirteenth aspect, characterized in that further comprising the steps for image reconstructing tomograms from projection data acquired in the same scanning position and synthesizing a new tomogram by subjecting tomograms having undergone image reconstruction from projection data on the same reconstruction plane in different scanning positions to interpolation or weighted addition on a pixel-by-pixel basis.

By the X-ray CT imaging method according to the eighteenth aspect, as it synthesizes tomograms on the basis of projection data acquired in different scanning positions and synthesizes them at the projection data stage, has an advantage of obtaining tomograms of a plurality of types.

According to its nineteenth aspect, the invention provides the X-ray CT imaging method according to the eighteenth aspect, characterized in that further comprising image reconstructing tomograms on one or more reconstruction planes from projection data acquired in the same scanning position and synthesizing a new tomogram by subjecting tomograms having undergone image reconstruction from projection data on a reconstruction plane contained in a prescribed range in the z-axis direction in the same scanning position and in different scanning positions to interpolation or weighted addition on a pixel-by-pixel basis.

By the X-ray CT imaging method according to the nineteenth aspect, as tomograms are synthesized on the basis of projection data acquired in different scanning positions and they are synthesizes at the projection data stage, provides an advantage of obtaining tomograms of a plurality of types. Also, as not only tomograms on the same reconstruction plane but also tomograms on a reconstruction plane contained in a prescribed range in the z direction are synthesized, tomograms of a prescribed width in the z direction can be subjected to image reconstruction.

According to its twentieth aspect, the invention provides the X-ray CT imaging method according to the eighteenth aspect or the nineteenth aspect, characterized in that the interpolation coefficient for the interpolation or the weighted addition coefficient for the weighted addition is determined on the basis of the geometrical positions and directions of the X-ray beams passing the pixels of the tomograms to be subjected to interpolation or weighted addition on the pixel-by-pixel basis.

By the X-ray CT imaging method according to the twentieth aspect, as the interpolation coefficient or the weighted addition coefficient is controlled according to the geometrical positions and directions of the X-ray beams, picture quality can be improved by reducing artifacts.

The X-ray CT apparatus and the X-ray CT imaging method according to the invention can help improve unevenness of picture quality dependent on the position of the reconstructed plane when conventional scanning or cine-scanning by an X-ray CT apparatus having a multi-row X-ray detector is performed in consecutive different scanning positions in the body axis direction (z-axis direction) of a subject.

### BRIEF DESCRIPTION OF THE DRAWINGS

FIG. 1 is a configurational block diagram showing an X-ray CT apparatus pertaining to Embodiment 1.

FIG. 2 is a diagram illustrating the geometrical arrangement of the X-ray tube and the multi-row X-ray detector as viewed in the z-axis direction.

FIG. 3 is a diagram illustrating the geometrical arrangement of the X-ray tube and the multi-row X-ray detector as viewed in the x-axis direction.

FIG. 4 is a flow chart outlining the operation of the X-ray CT apparatus pertaining to Embodiment 1.

FIG. 5 is a diagram illustrating the scanning position and the X-ray beam pertaining to Embodiment 1.

FIG. 6 is a diagram illustrating the row-directional filter coefficient.

FIG. 7 is a diagram illustrating a state in which the slice thickness is greater on the peripheries than at the center of a reconstruction area.

FIG. 8 is a diagram illustrating a row-directional filter coefficient varying from channel to channel.

FIG. 9 is a diagram illustrating a state in which the slice thickness is uniform whether at the center or on the peripheries a reconstruction area.

FIG. 10 is a diagram illustrating a row-directional filter coefficient for reducing the slice thickness.

FIG. 11 is a flow chart showing details of three-dimensional back-projection processing pertaining to Embodiment 1.

FIGS. 12a and 12b are conceptual diagrams showing a state in which a pixel row on a reconstruction plane P is projected in the X-ray transmitting direction.

FIG. 13 is a conceptual diagram showing a line of projecting on a detector face the pixel row on the reconstruction plane P.

FIGS. 14a and 14b are conceptual diagrams showing an X-ray beam passing the same pixel g on the same reconstruction plane P though differing in scanning position.

FIGS. 15a and 15b are conceptual diagrams showing an X-ray beam passing the same pixel g and the vicinities of the pixel g on the same reconstruction plane P though differing in scanning position.

FIG. 16 is a conceptual diagram showing pixel data Dr on the reconstruction plane P at a view angle of view=0°.

FIG. 17 is a conceptual diagram showing back-projected pixel data D2 on the reconstruction plane P at a view angle of view=0°.

FIG. 18 is a diagram illustrating a state in which back-projection data D3 are obtained by subjecting the back-projected pixel data D2 to all-view addition on a pixel-by-pixel basis.

FIG. 19 is a conceptual diagram showing a circular reconstruction plane P.

FIGS. 20a, 20b, 20c, and 20d are conceptual diagrams describing effects pertaining to Embodiment 1.

FIG. 21 is a diagram illustrating scanning positions and the expansion of the X-ray beam pertaining to Embodiment 2.

FIG. 22 is a diagram illustrating scanning positions and the expansion of the X-ray beam pertaining to Embodiment 3.

FIG. 23 is a diagram illustrating scanning positions and the expansion of the X-ray beam pertaining to Embodiment 4.

FIG. 24 is a flow chart of an X-ray CT imaging method pertaining to Embodiment 5.

FIG. 25 is a detailed flow chart of three-dimensional back-projection processing pertaining to Embodiment 5.

FIG. 26 is a conceptual diagram describing effects pertaining to Embodiment 5.

FIG. 27 is a flow chart of an X-ray CT imaging method pertaining to Embodiment 6.

FIG. 28 is a diagram illustrating scanning positions and the expansion of the X-ray beam pertaining to the first prior art case.

FIG. 29 is a conceptual diagram describing problems pertaining to the first prior art case.

FIG. 30 is another conceptual diagram describing problems pertaining to the first prior art case.

FIG. 31 is a diagram illustrating scanning positions and the expansion of the X-ray beam pertaining to the second prior art case.

### DETAILED DESCRIPTION OF THE INVENTION

The present invention will be described in further detail below with reference to illustrated modes for carrying it out. Incidentally, the invention is not to be limited by the following description.

### Embodiment 1

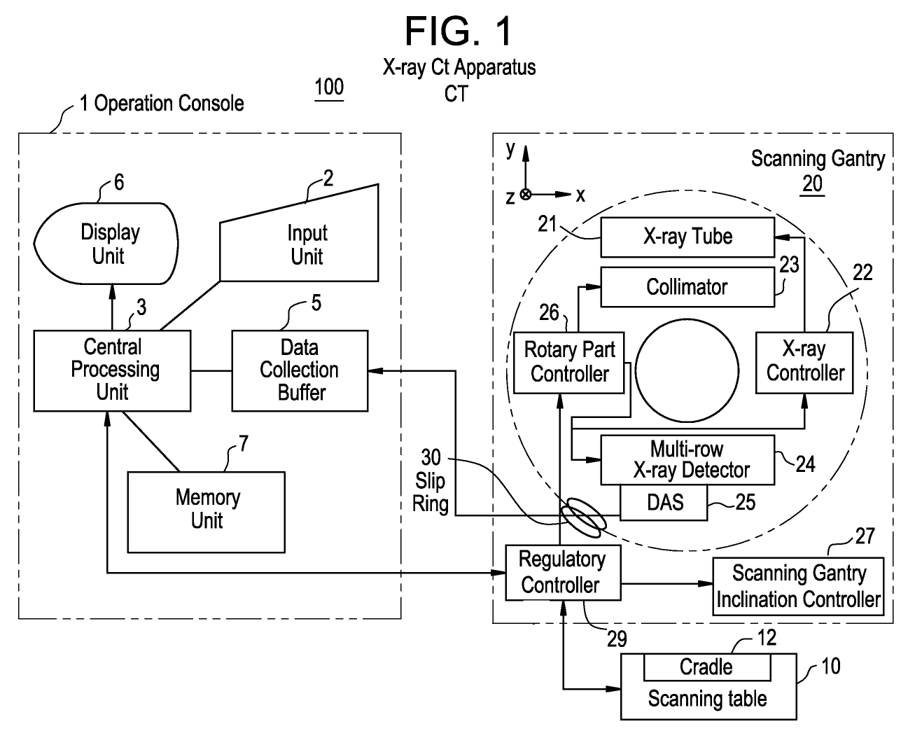

FIG. 1 is a configurational block diagram showing an X-ray CT apparatus pertaining to Embodiment 1.

This X-ray CT apparatus 100 is equipped with an operation console 1, an scanning table 10 and a scanning gantry 20.

The operation console 1 is provided with an input unit 2 which accepts inputs by the operator, a central processing unit 3 which executes pre-treatments, image reconstruction processing, post-treatments and so forth, a data acquisition buffer 5 which acquires projection data acquired by the scanning gantry 20, a display unit 6 which displays tomograms reconstructed from projection data obtained by pre-treating acquired projection data, and a memory unit 7 which stores programs, data, projection data and X-ray tomograms.

The scanning table 10 is provided with a cradle 12 which brings a subject mounted thereover in and out through an opening in the scanning gantry 20. The cradle 12 is moved up and down and linearly by a motor built into the scanning table 10.

The scanning gantry 20 is provided with an X-ray tube 21, an X-ray controller 22, collimators 23, a multi-row X-ray detector 24, a DAS (Data Acquisition System) 25, a rotary part controller 26 which controls the X-ray tube 21 and other elements turning around the rotation center axis, a regulatory controller 29 which exchanges control signals and the like with the operation console 1 and the scanning table 10, and a slip ring 30 which transfers power, control signals and acquired data. The scanning gantry 20 can be inclined by about ±30° forward or backward by a scanning gantry inclination controller 27.

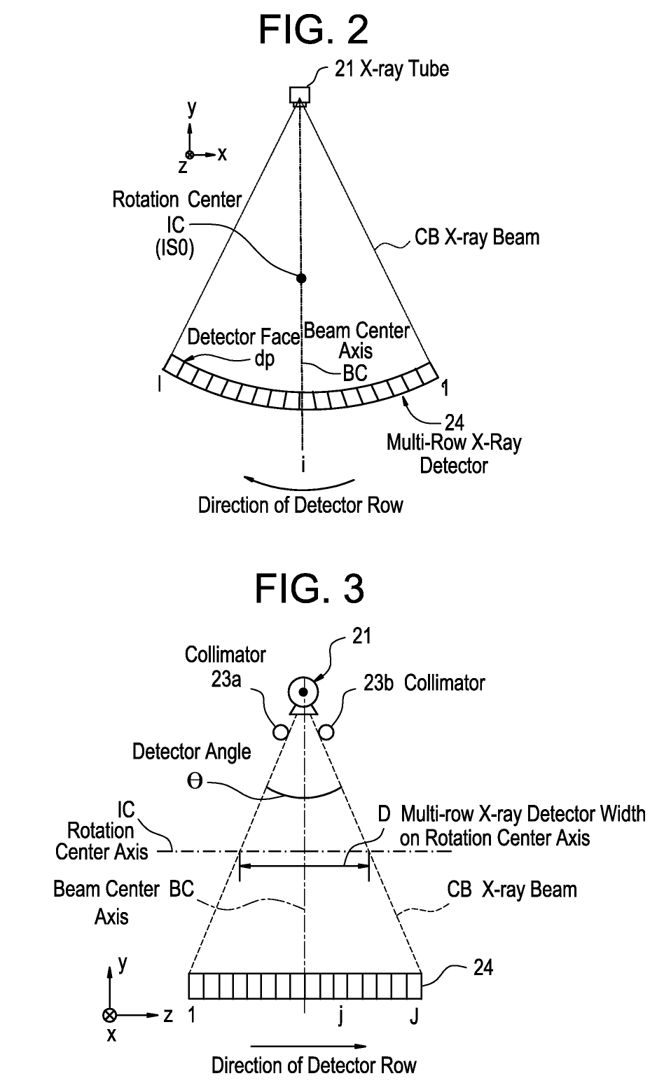

FIG. 2 and FIG. 3 are diagrams illustrating the geometrical arrangement of the X-ray tube 21 and the multi-row X-ray detector 24.

The X-ray tube 21 and the multi-row X-ray detector 24 turn around the rotation center axis IC. Where the vertical direction is supposed to be the y direction, the linearly transferred direction of the cradle 12 is supposed to be the z-axis direction, the direction orthogonal to the z-axis direction and the y-axis direction are supposed to the x-axis direction, and the inclination angle of the scanning gantry 20 is supposed to be 0°, the rotating plane of the X-ray tube 21 and the multi-row X-ray detector 24 is the xy plane.

The X-ray tube 21 generates an X-ray beam CB known as a cone beam. When the direction of the beam center axis BC, which is the center axis of the X-ray beam CB, is parallel to the y direction, the view angle is supposed to be 0°.

The multi-row X-ray detector 24 has first through J-th rows of detectors, where J=256 for instance. Further each row of detectors has first through T-th channels, where I=1024 for instance.

As shown in FIG. 3, the multi-row X-ray detector width D is the width of the multi-row X-ray detector 24 in the z-axis direction on the rotation center axis IC when the multi-row X-ray detector 24 is viewed from the focus of the X-ray tube 21. Further, the detector angle θ is the angle of the multi-row X-ray detector 24 in the z-axis direction when the multi-row X-ray detector 24 is viewed from the focus of the X-ray tube 21.

A collimator 23a defines the opening edge of the forward side of the X-ray beam CB in the z-axis direction, and a collimator 23b defines the opening edge of the backward side of the X-ray beam CB in the z-axis direction.

Projected data which are irradiated with X-rays and acquired undergo A/D conversion from the multi-row X-ray detector 24 to the DAS 25, and are inputted to the data acquisition buffer 5 via the slip ring 30.

The projection data inputted to the data acquisition buffer 5 undergo image reconstruction by the central processing unit 3 according to a program stored in the memory unit 7, and are converted into a tomogram. The tomogram is displayed on the display unit 6.

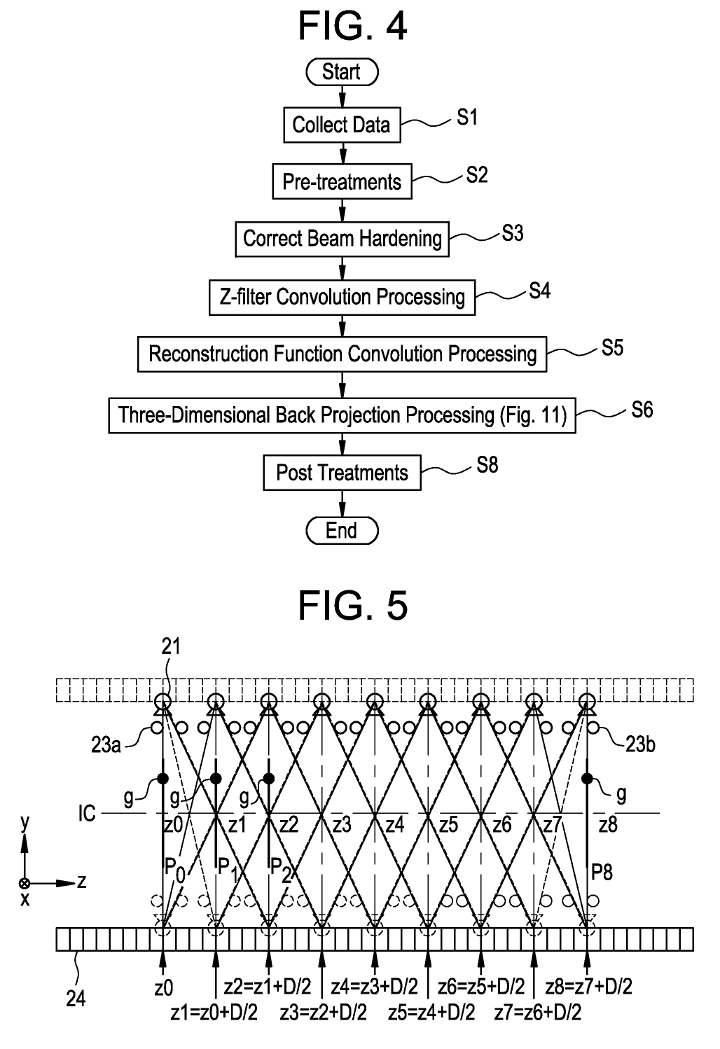

FIG. 4 is a flow chart outlining the operation of the X-ray CT apparatus 100.

At step S1, conventional scanning or cine-scanning is performed in consecutive different scanning positions in the z-axis direction to acquire projection data.

For instance in the scanning position z0 shown in FIG. 5, the X-ray tube 21 and the multi-row X-ray detector 24 are turned round the rotation center axis IC to acquire projection data comprising projection data D0 (view, j, i) represented by a view angle view, a detector row number j and a channel number i to which the scanning position z0 is added. Hereupon, the collimator 23a is controlled to make the opening edge of the forward side of the X-ray beam CB in the z-axis direction “z0−δ”, (δ is 0 or an appropriately small positive number), and the collimator 23b is controlled to make the opening edge of the backward side of the X-ray beam CB in the z-axis direction “z2+D/2+δ”. As a result, the expanding angle of the X-ray beam CB becomes θ/2 or substantially θ/2 relative to the detector angle θ.

Next, the cradle 12 is controlled for a linear transfer by D/2, and the X-ray tube 21 and the multi-row X-ray detector 24 are turned round the rotation center axis IC in the scanning position z1 (=z0+D/2) to acquire projection data comprising projection data D0 (view, j, i) represented by a view angle view, a detector row number j and a channel number i to which the scanning position z1 is added. Hereupon, the collimator 23a is controlled to make the opening edge of the forward side of the X-ray beam CB in the z-axis direction “z1−D/4−δ” on the rotation center axis IC, and the collimator 23b is controlled to make the opening edge of the backward side of the X-ray beam CB in the z-axis direction “z1+D/2+δ” on the rotation center axis IC.

Then, the cradle 12 is controlled for a linear transfer by D/2, and the X-ray tube 21 and the multi-row X-ray detector 24 are turned round the rotation center axis IC in the scanning position z2 (=z1+D/2) to acquire projection data comprising projection data D0 (view, j, i) represented by a view angle view, a detector row number j and a channel number i to which the scanning position z2 is added. Hereupon, the collimator 23a is controlled to make the opening edge of the forward side of the X-ray beam CB in the z-axis direction “z2−D/2−δ” on the rotation center axis IC, and the collimator 23b is controlled to make the opening edge of the backward side of the X-ray beam CB in the z-axis direction “z2+D/2+δ” on the rotation center axis IC.

Next, as in the scanning position z2, the cradle 12 is linearly transferred by D/2 at a time, and projection data D0 are acquired by performing conventional scanning or cine-scanning in the scanning positions z2, Z3, z4, z5 and Z6.

Then, the cradle 12 is controlled for a linear transfer by D/2, the X-ray tube 21 and the multi-row X-ray detector 24 are turned round the rotation center axis IC in the scanning position z7 (=z6+D/2) to acquire projection data comprising projection data D0 (view, j, i) represented by a view angle view, a detector row number j and a channel number i to which the scanning position z7 is added. Hereupon, the collimator 23a is controlled to make the opening edge of the forward side of the X-ray beam CB in the z-axis direction “z7−D/2−δ” on the rotation center axis IC, and the collimator 23b is controlled to make the opening edge of the backward side of the X-ray beam CB in the z-axis direction “z8+D/4+δ” on the rotation center axis IC.

Next, the cradle 12 the cradle 12 is controlled for a linear transfer by D/2, the X-ray tube 21 and the multi-row X-ray detector 24 are turned round the rotation center axis IC in the scanning position z8 (=z7+D/2) to acquire projection data comprising projection data D0 (view, j, i) represented by a view angle view, a detector row number j and a channel number i to which the scanning position z8 is added. Hereupon, the collimator 23a is controlled to make the opening edge of the forward side of the X-ray beam CB in the z-axis direction “z8−D/2−δ” on the rotation center axis IC, and the collimator 23b is controlled to make the opening edge of the backward side of the X-ray beam CB in the z-axis direction “z8+δ” on the rotation center axis IC.

Referring back to FIG. 4, at step S2, projection data D0 (view, j, i) acquired in the scanning positions z0 through z8 are subjected to pre-treatments including offset correction, logarithmic conversion, X-ray dose correction and sensitivity correction to obtain projection data Din (view, j, i).

At step S3, the projection data Din (view, j, i) acquired in the scanning positions z0 through z8 and having undergone pre-treatments are subjected to beam hardening. The beam hardening is represented by, for instance, the following polynomial, where B0, B1 and B2 are beam hardening coefficients.

Dout(view,j,i)=Din(view,j,i)×(B₀(ji)+B₁(j,i)×Din(view,j,i)+B₂(j,i)×Din(view,j,i)²)

Since each detector row of the multi-row X-ray detector 24 can be subjected to independent beam hardening correction here, if the tube voltages of data acquisition lines are different under the scanning conditions, differences in characteristics among the detector rows can be compensated for.

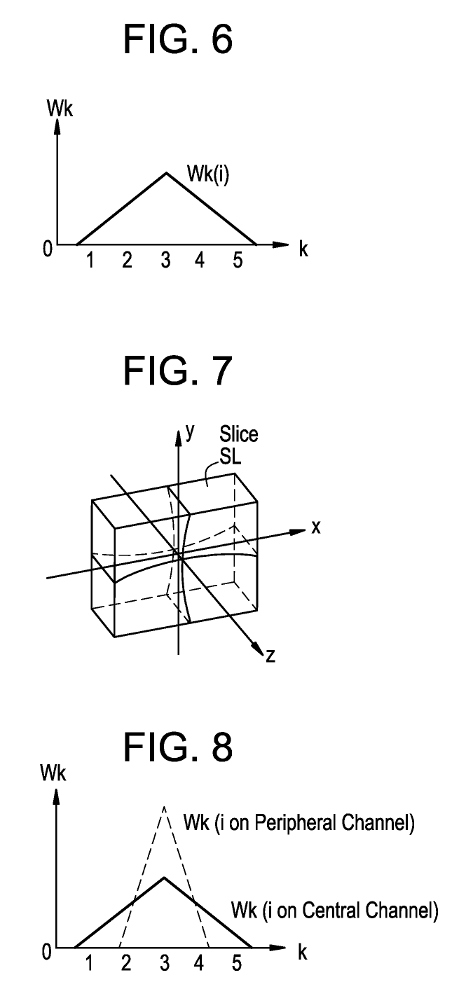

At step S4, the projection data Dout (view, j, i) acquired in the scanning positions z0 through z8 and having undergone pre-treatments and beam hardening correction are subjected to filter convolution, by which filtering in the z direction (row direction) is applied. Thus, the projection data Dout (view, j, i) are multiplied by a row-directional filter coefficient Wk(i) in a row direction, such as the one shown in FIG. 6, to figure out projection data Dcor (view, j, i).

$$Dcor ( view , j , i ) = {\sum }_{k = 1}^{5} ( Dout ( view , j + k - 3 , i ) \times Wk ( i ) )$$

$$where {\sum }_{k = 1}^{5} ( Wk ( i ) ) = 1$$

$$Dout ( view , - 1 , i ) = Dout ( view , 0 , i ) = Dout ( view , 1 , i )$$

$$Dout ( view , J + 1 , i ) = Dout ( view , J + 2 , i ) = Dout ( view , J , i )$$

$$are obtained .$$

Further by varying the row-directional filter coefficient from channel to channel, the slice thickness can be controlled according to the distance from the reconstruction center.

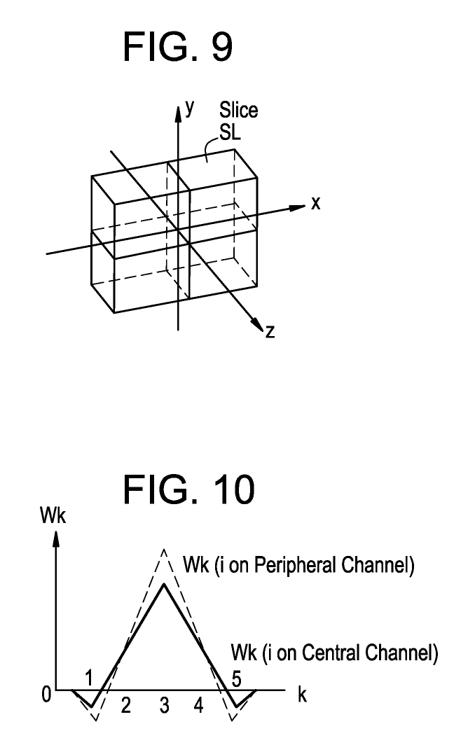

As is seen from a slice SL shown in FIG. 7, generally the slice thickness is greater on the peripheries than at the center of reconstruction. In view of this, as shown in FIG. 8, by using a row-directional filter coefficient Wk (i of central channels) which extensively varies the width for central channels and a row-directional filter coefficient Wk (i of peripheral channels) which narrowly varies the width for peripheral channels, a slice SL of substantially uniform slice thickness both at the center and on the peripheries of reconstruction can be obtained as shown in FIG. 9.

Slightly increasing slice thickness by the row-directional filter coefficient Wk(i) results in improvement both in artifact and noise aspects. This enables the extent of artifact improvement and that of noise improvement to be controlled. In other words, the picture quality of even a tomogram having undergone three-dimensional image reconstruction can be controlled.

By making the row-directional filter coefficient Wk(i) a deconvolution filter as shown in FIG. 10, tomograms of a small slice thickness can also be realized.

Referring back to FIG. 4, convolution of the reconstructive function is processed at Step S5. Thus, the result of Fourier transform is multiplied by the reconstructive function to achieve inverse Fourier transform. Projected data after the convolution of reconstructive function being represented by Dr (view, j, i), the reconstructive function by Kernel (j) and convolution computing by *, the processing to convolute the reconstructive function can be expressed in the following way.

Dr(view,j,i)=Dcor(view,j,i)*Kernel(j)

Since reconstructive function convolution can be processed independently on each detector row by using an independent reconstructive function Kernel(j), differences in noise characteristics and resolution characteristics among detector rows can be compensated for.

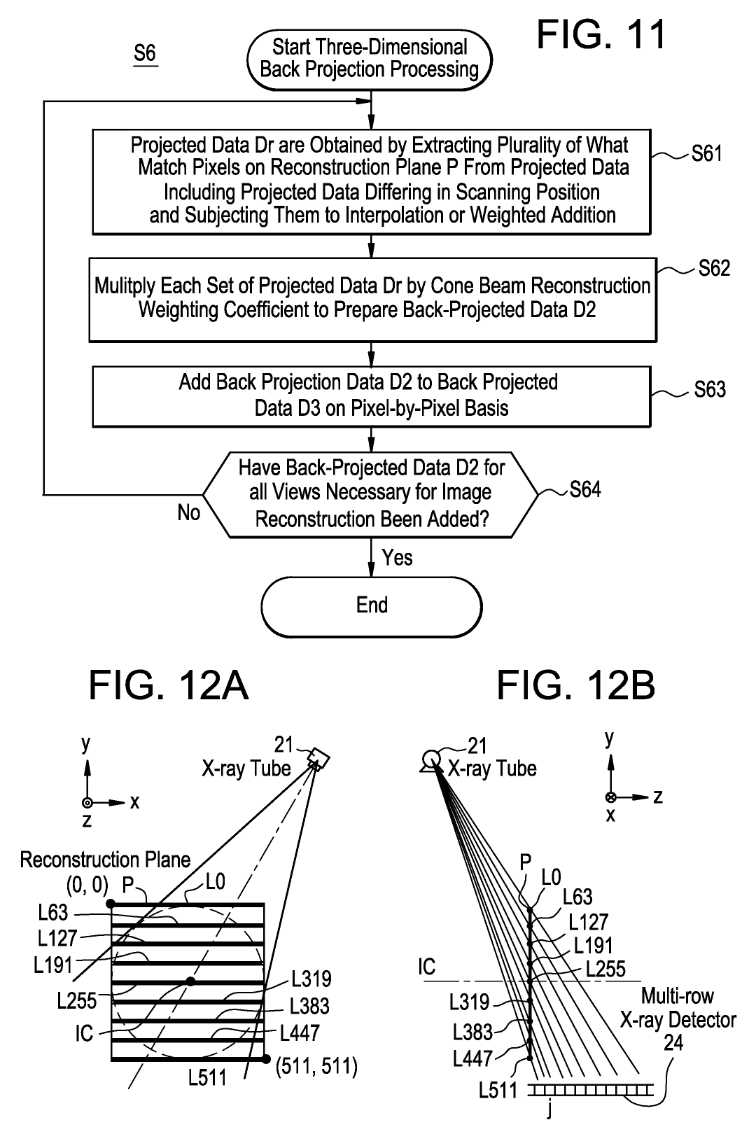

At step S6, the projection data Dr (view, j, i) are subjected to three-dimensional back-projection processing to figure out back-projection data D3 (x, y). This three-dimensional back-projection processing will be described afterwards with reference to FIG. 11.

At step S8, the back-projection data D3 (x, y) are subjected to post-treatments including image filter convolution and CT value conversion to obtain a tomogram.

In the image filter convolution processing, with the data having gone through image filter convolution processing being represented by D4 (x, y) and the image filter by Filter (x, y), the following holds:

D4(x,y)=D3(x,y)*Filter(x,y)

Then, since image filter convolution can be processed independently in each slicing position of the tomogram, differences in noise characteristics and resolution characteristics among slice positions can be compensated for.

FIG. 11 is a flow chart showing details of the three-dimensional back-projection processing (step S6 in FIG. 4).

At step S61, one view out of all the views necessary for tomogram reconstruction (namely views corresponding to 360° or views “corresponding to 180°+fan angle”) is taken note of, and a plurality of sets of projection data of the noted view corresponding to each pixel of a reconstruction plane P out of projection data also including projection data differing in scanning position are extracted and subjected to interpolation or weighted addition to obtain projection data Dr.

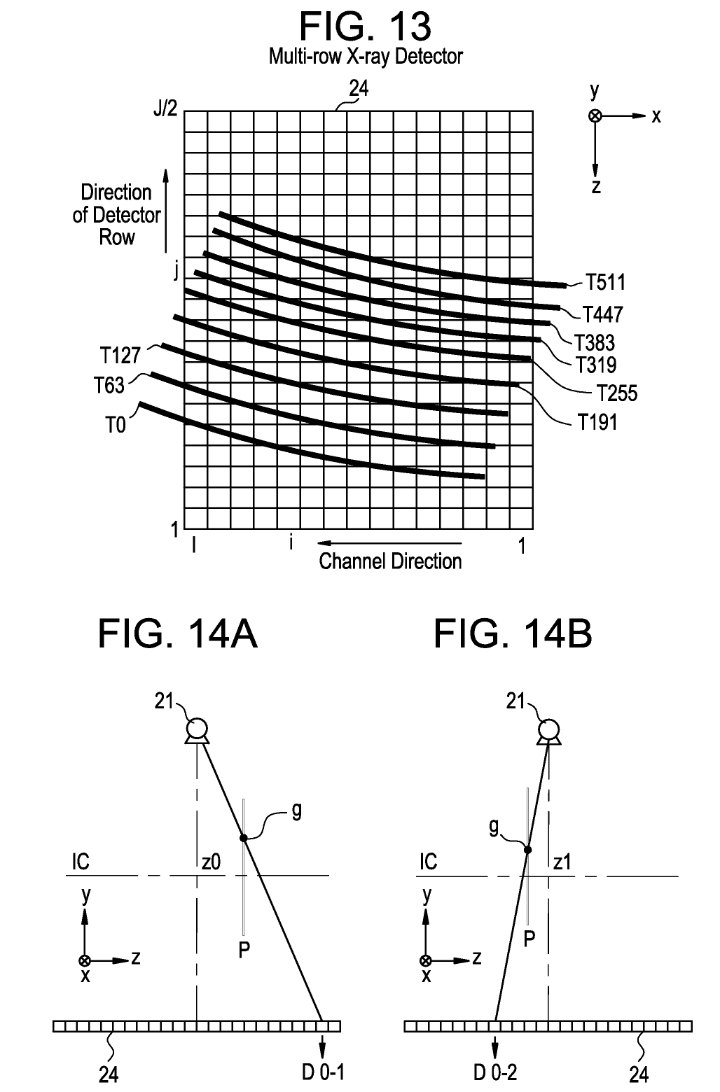

As shown in FIG. 12, in an exemplary case of a square reconstruction plane P having 512×512 pixels parallel to the xy plane in which a pixel row of y=0 parallel to the x axis is represented by L0, a pixel row of y=63 by L63, a pixel row of y=127 by L127, a pixel row of y=191 by L191, a pixel row of y=255 by L255, a pixel row of y=319 by L319, a pixel row of y=383 by L383, a pixel row of y=447 by L447, and a pixel row of y=511 by L511, projection data D0 on lines T0 through T511 resulting from the projection of these pixel rows L0 through L511 on the face of the multi-row X-ray detector 24 in the transmitting direction of the X-ray beam in a certain scanning position as shown in FIG. 13 are extracted. Incidentally, where part of a line goes out of the multi-row X-ray detector 24, like the line T0 in FIG. 13, the corresponding projection data D0 are reduced to “0”. Or where part of a line goes out of the direction of the detector row, projection data D0 are figured out by extrapolation. Projected data D0 of the detector rows L0 through L511 are extracted by applying this procedure to different scanning positions. Subjecting the plurality of sets of extracted projection data D0 to interpolation or weighted addition will give the projection data Dr of the detector rows L0 through L511. If, for instance, a plurality of sets of projection data D0_1 and D0_2 matching the X-ray beam passing the pixel g are extracted as shown in FIG. 14, the following will hold:

Dr=k1·D0_{—}1+k2·D0_{—}2

Where k1 and k2 are interpolation coefficients or weighted addition coefficients, which are determined on the basis of the geometrical positions and directions of the X-ray beams passing the pixels matching the sets of projection data D0 to be subjected to interpolation or weighted addition. Incidentally, k1+k2=1 is supposed.

Whereas the transmitting direction of an X-ray beam is determined by the X-ray focus of the X-ray tube 21 and the geometrical positions of pixels and of the multi-row X-ray detector 24, since the z coordinates of the projection data D0 (view, j, i) are known, the transmitting direction of the X-ray beam can be accurately figured out even for projection data D0 (view, j, i) under acceleration or deceleration.

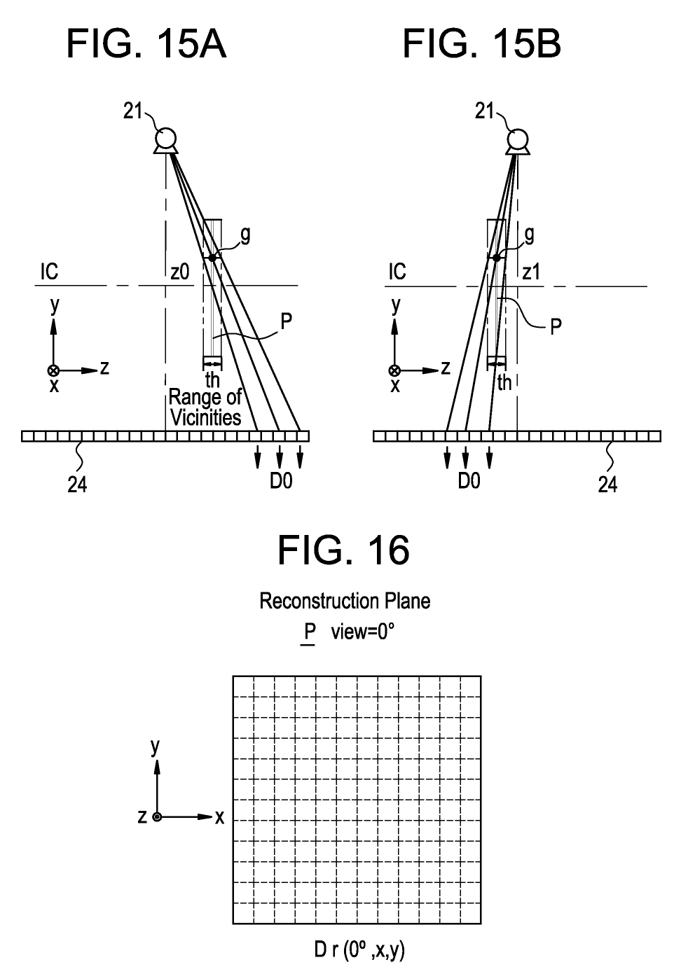

To add, as shown in FIG. 15, a plurality of sets of projection data D0 which are projection data acquired in the same scanning position and different scanning positions and match the X-ray beam passing the same pixel on reconstruction plane P or a nearby range th in the z direction centering that pixel g may be subjected to interpolation or weighted addition to synthesize projection data Dr image reconstruction.

In this way, as shown in FIG. 16, projection data Dr (view, j, i) matching each pixel on the reconstruction plane P can be obtained.

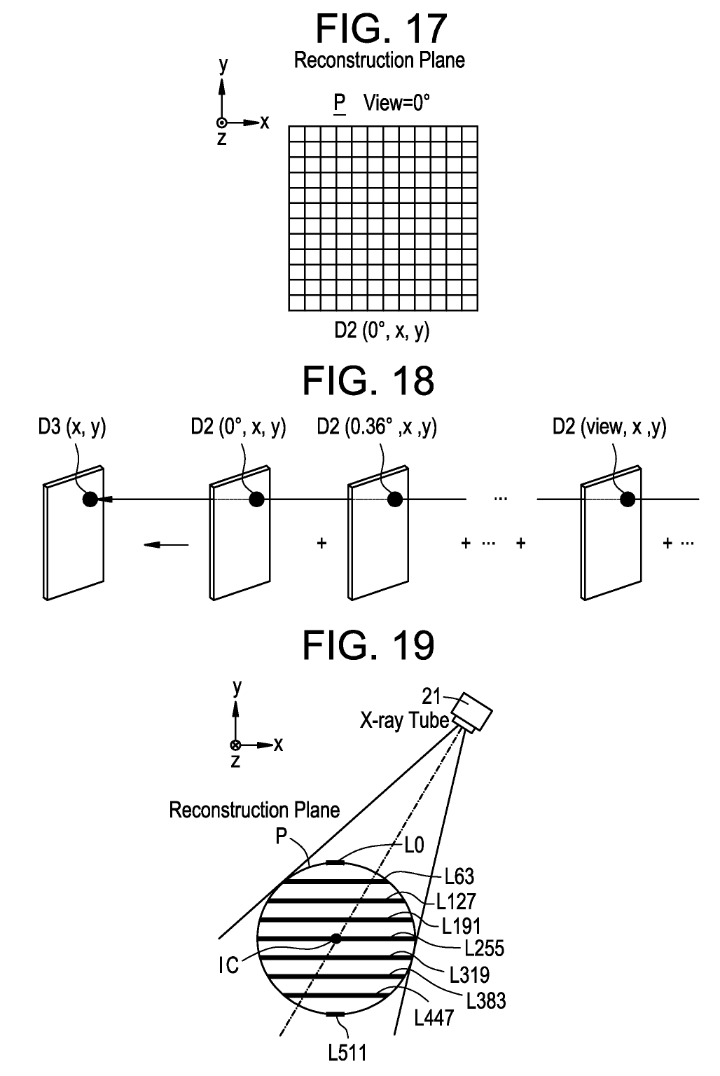

Referring back to FIG. 11, at step S62, projection data Dr (view, x, y) are multiplied by a cone beam by a reconstruction weighting coefficient to prepare projection data D2 (view, x, y) shown in FIG. 17.

The cone beam reconstruction weighting coefficient here is as described below.

In the case of fan beam image reconstruction, where an angle which a straight line linking the focus of the X-ray tube 21 and a pixel g (x, y) on the reconstruction plane P (on the xy plane) in view=βa forms with the center axis Bc of the X-ray beam is represented by γ and the view opposite it is view=βb: the following holds:

(b=(a+180(−2(

The angle formed by the X-ray beam passing pixel g (x, y) on the reconstruction plane P and the angle formed by the X-ray beam opposite it on the reconstruction plane P are represented by (a and (b, they are added with multiplication by the cone beam reconstruction weighting coefficients (a and (b dependent on them to figure out the back-projection data D2 (0, x, y).

D2(0,x,y)=(a·D2(0,x,y)_{—}a+(b·D2(0,x,y)_{—}b

Here, D2 (0, x, y)_a are supposed to be the projection data in the view (a and, D2 (0, x, y) b, the projection data in the view (b.

Incidentally, the sum of the respective cone beam reconstruction weighting coefficients ωa and ωb of the X-ray beam and of the X-ray beam opposite it is ωa+ωb=1.

By addition with multiplication by the cone beam reconstruction weighting coefficients ωa and ωb as stated above, the cone beam angle artifacts can reduced.

For instance, what are obtained by the following equations can be used as the cone beam reconstruction weighting coefficients ωa and ωb.

Where f( ) represents a function and the fan beam angle is γmax:

ga=f(γ max,αa,βa)

gb=f(γ max,αb,βb)

xa=2·ga^{q}/(ga^{q}+gb^{q})

xb=2·gb^{q}/(ga^{q}+gb^{q})

ωa=xa²·(3−2xa)

ωb=xb²·(3−2xb)

(q=1 is supposed, for instance)

Where what takes the greater value of f( ) is represented by a function max [ ], the following holds.

ga=max[0,{(π/2+γ max)−|βa|}]·|tan(αa)|

gb=max[0,{(π/2+γ max)−|βb|}]·|tan(αb)|

In the case of fan beam image reconstruction, the projection data Dr of each pixel on the reconstruction plane P is further multiplied by a distance coefficient. The distance coefficient is (r1/r0)² where the distance from the focus of the X-ray tube 21 to the detector row j, channel i of the multi-row X-ray detector 24 matching the projection data Dr is represented by r0 and the distance from the focus of the X-ray tube 21 to the pixel on the reconstruction plane P matching the projection data Dr is represented by r1.

In the case of parallel beam image reconstruction, the projection data Dr of each pixel on the reconstruction plane P need to be multiplied only by a cone beam reconstruction weighting coefficient.

At step S63, as shown in FIG. 18, projection data D2 (view, x, y) are added pixel by pixel to back-projection data D3 (x, y) cleared in advance.

At step S64, with respect to all the views needed for tomogram reconstruction (namely views corresponding to 360° or views “corresponding to 180°+fan angle”), steps S61 through S63 are repeated, and back-projection data D3 (x, y) are obtained as shown in FIG. 18.

Incidentally, as shown in FIG. 19, the reconstruction plane P may be a circular area.

The X-ray CT apparatus 100 of Embodiment 1 provides the following effects.

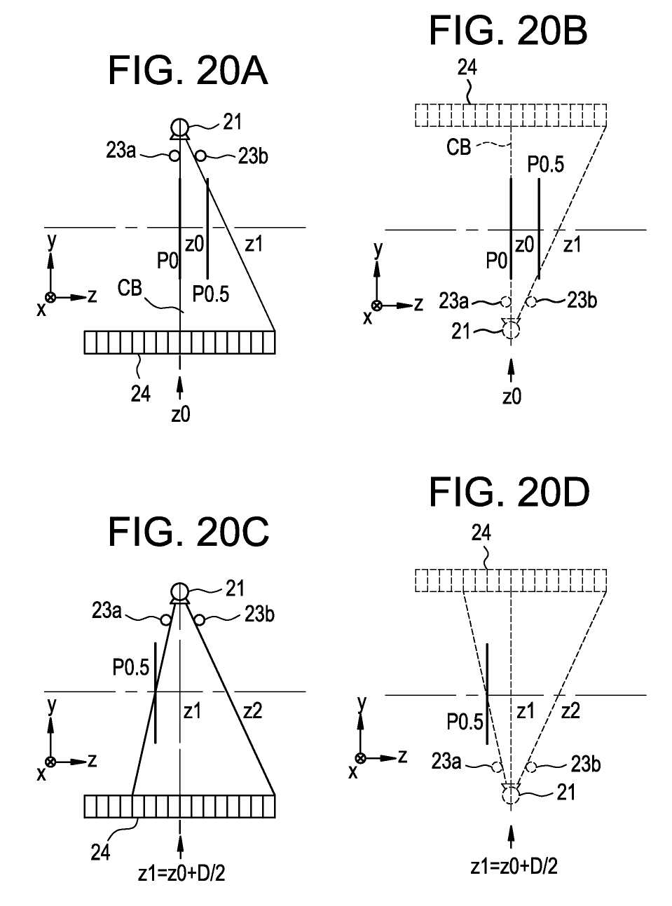

(1) As shown in FIGS. 20(a) and 20(b), projection data can be obtained in every view angle for any pixel even on an end reconstruction plane P0, and the inclination of the X-ray beam CB relative to the reconstruction plane P0 is reduced. As a result the picture quality of the tomogram even on an end reconstruction plane P0 is made sufficiently high.

As shown in FIGS. 20(a) through 20(d), since the interval between the scanning position z0 and the scanning position z1 is made D/2, the inclination of the X-ray beam CB relative to the reconstruction plane P0.5 positioned between the scanning position z0 and the scanning position z1 can be made small and uniform with little fluctuations. As a result, the picture quality of the tomogram on the reconstruction plane P0.5 positioned between the scanning position z0 and the scanning position z1 can be improved.

Similarly, the picture quality of the tomograms on the other end reconstruction plane P8 and of tomograms on other reconstruction planes positioned between one scanning position and another scanning position can also be improved.

Thus, unevenness of picture quality dependent on the position of the reconstruction plane can be improved.

(2) As shown in FIGS. 20(a) and 20(b), since the width of the X-ray beam CB is narrowed in one end scanning position z0, any wastefully irradiated area can be reduced. Similarly, since the width of the X-ray beam CB is also narrowed in the other end scanning position z8, any wastefully irradiated area there can be reduced. An increase in irradiation due to narrowing the interval between one scanning position and another to not more than D can be avoided by restraining the X-ray dose and the X-ray tube current.

(3) Since projection data acquired in different scanning positions are synthesized at the projection data stage, only one step of image reconstruction computing is needed.

Incidentally, the image reconstruction method here may be the usual three-dimensional image reconstruction method according to the already known Feldkamp method. Further, the three-dimensional image reconstruction method proposed in JP-A No. 334188/2003, JP-A No. 41675/2004, JP-A No. 41674/2004, JP-A No. 73360/2004, JP-A No. 159244/2003 or JP-A No. 41675/2004 may be used as well.

Also according to Embodiment 1, picture quality fluctuations due to differences in the X-ray cone angle or other causes can be adjusted by convoluting row-directional (z-direction) filters differing in coefficient over different detector rows, and uniform slice thickness and picture quality in terms of artifacts and noise are realized, but similar effects can also be achieved in some other way.

Further, though the interval between one scanning position and another is reduced to D/2, any other interval not greater than D can achieve picture quality improvement over the conventional level.

Also, though the X-ray beam is prevented from widening both forward and backward in the linear transfer direction beyond the range in which projection data D0 are to be acquired according to Embodiment 1, the range of irradiation can be narrowed by preventing from widening either forward or backward.

Further, an X-ray CT apparatus in which an X-ray area detector, typically a flat panel, is used as a multi-row X-ray detector in place of the multi-row X-ray detector 24 used in Embodiment 1, also permits application of the present invention.

### Embodiment 2

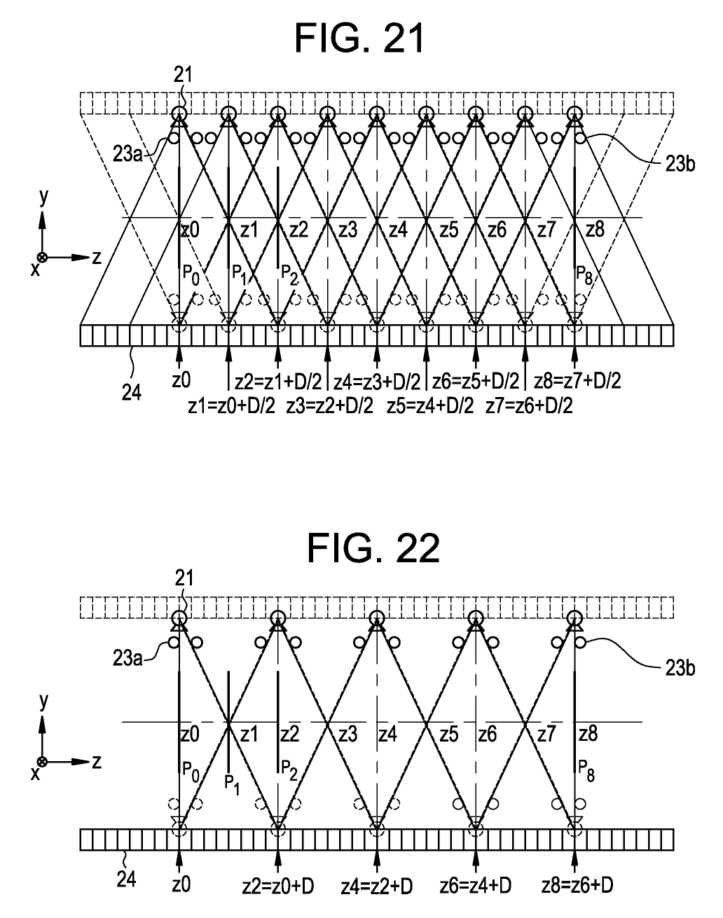

It is also possible to keep the width of the X-ray beam at D as in the conventional practice and use the same conditions as in Embodiment 1 in other respects as shown in FIG. 21.

In Embodiment 2 as well, unevenness in picture quality dependent on the position of the reconstruction plane can be improved. Incidentally, an increase in irradiation can be avoided by restraining the X-ray dose and the X-ray tube current.

It is also possible to keep the interval between one scanning position and another at D as in the conventional practice and prevent the X-ray beam from widening both forward and backward in the linear transfer direction beyond the range in which projection data D0 are to be acquired as shown in FIG. 22.

Embodiment 3 can also help improve the picture quality of the tomogram at both ends. The range of irradiation can be reduced, too.

### Embodiment 4

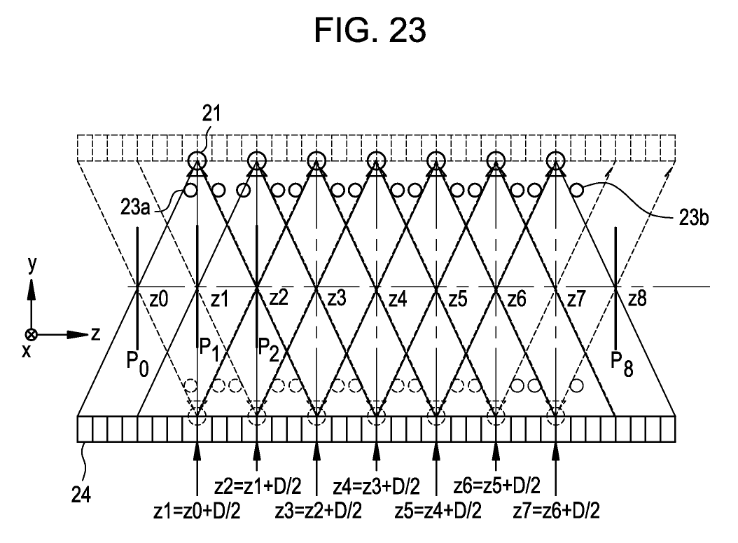

It is also possible to keep the width of the X-ray beam at D as in the conventional practice and keep the interval between one scanning position and another at not more than D (exactly or approximately D/2 in FIG. 22) as shown in FIG. 23.

Embodiment 4 can also help improve the picture quality of the tomogram positioned between one scanning position and another. Incidentally, by restraining the X-ray dose and the X-ray tube current, an increase in irradiation due to keeping the interval between one scanning position and another at D can be avoided.

### Embodiment 5

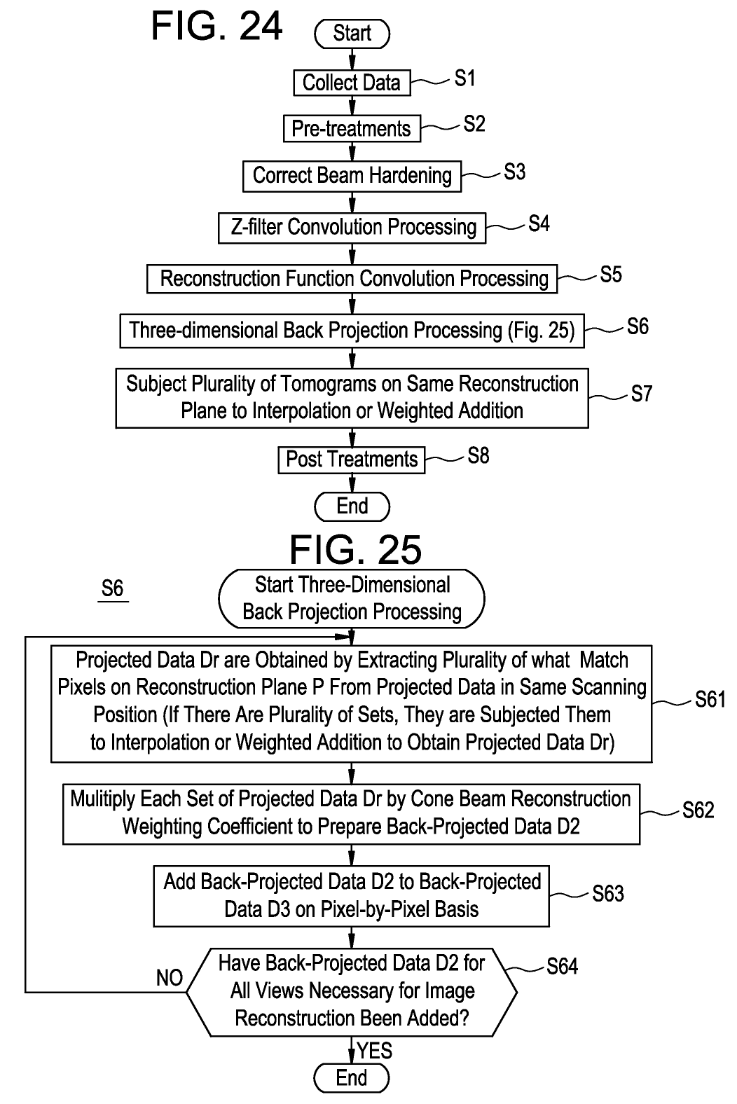

FIG. 24 is a flow chart of an X-ray CT imaging method pertaining to Embodiment 5.

As compared with the flow chart of the X-ray CT imaging method pertaining to shown in FIG. 4, step S6 in FIG. 4 is replaced by step S6′ here and step S7 is added. Other steps are the same. Therefore, only step S6′ and S7 will be described.

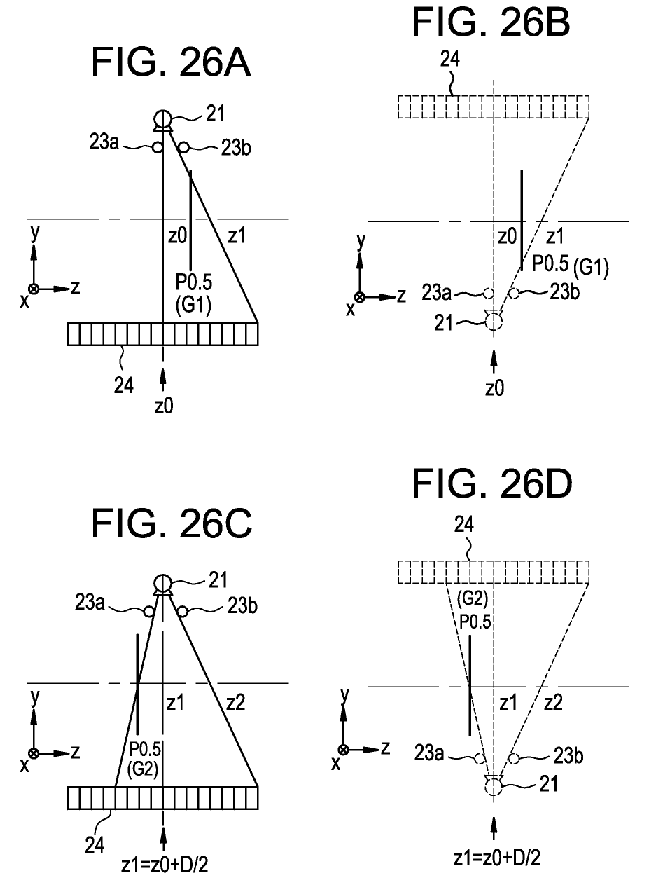

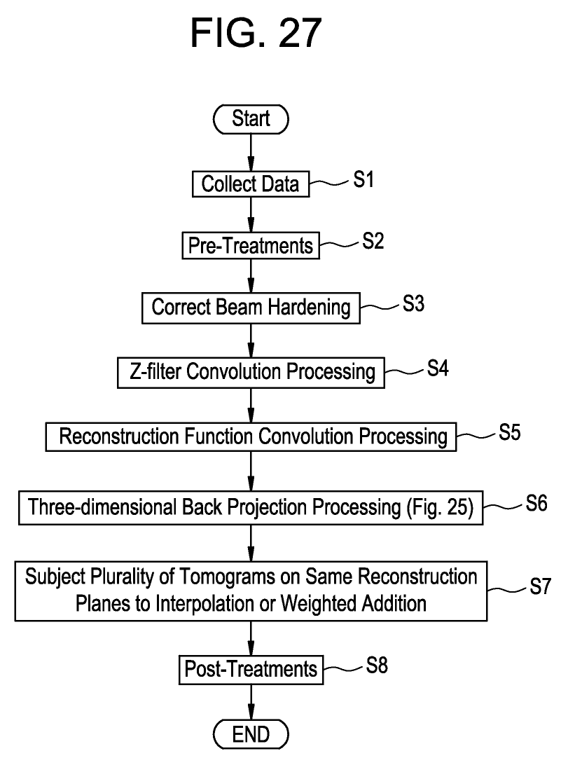

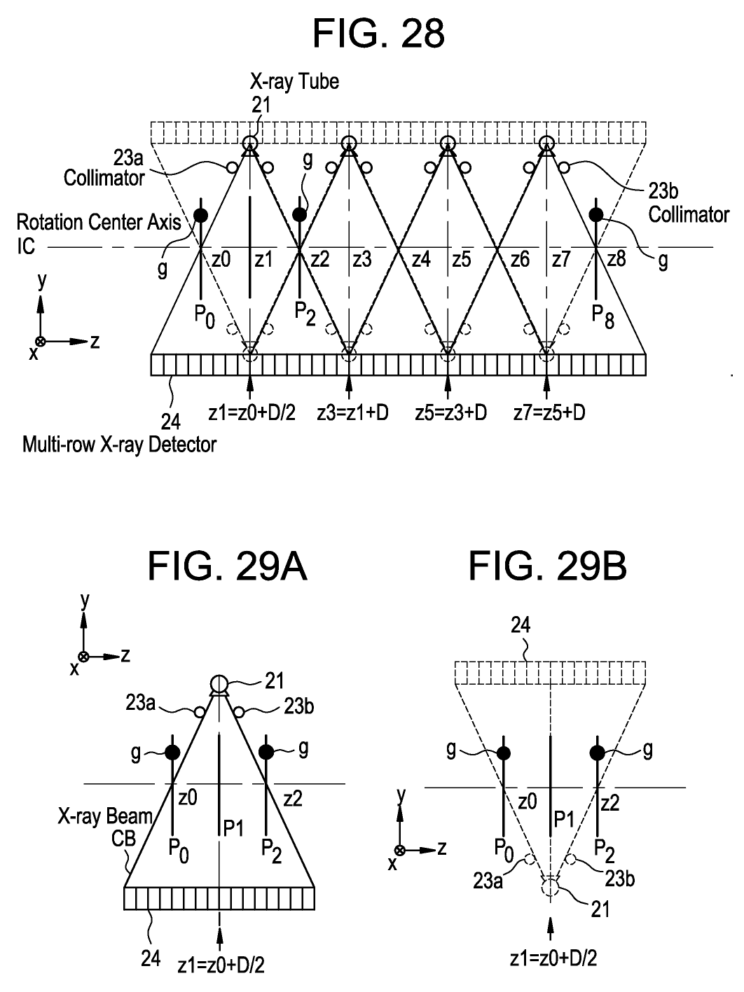

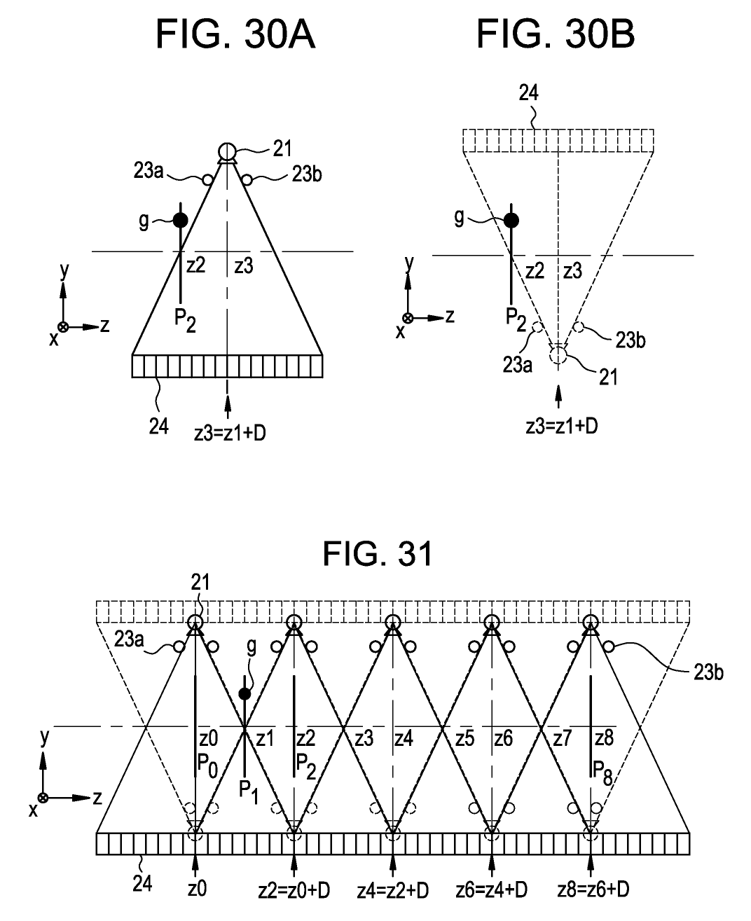

FIG. 25 is a detailed flow chart of step S6′ (three-dimensional back-projection processing).

As compared with the flow chart of three-dimensional back-projection processing of Embodiment 1 shown in FIG. 11, step S61 in FIG. 11 is replaced by step S61′ here. Other steps are the same. Therefore, only step S61′ will be described.

At step S61′, one view out of all the views necessary for tomogram reconstruction (namely views corresponding to 360° or views “corresponding to 180°+fan angle”) is taken note of, and a plurality of sets of projection data of the noted view corresponding to each pixel of a reconstruction plane P out of projection data of the same scanning position are extracted and subjected to interpolation or weighted addition to obtain projection data Dr.

Thus, though projection data Dr are obtained at step S61 in FIG. 11 by projection data extracted from projection data also including those differing in scanning position and extracted projected are interpolation or subjected to interpolation or weighted addition to obtain projection data Dr, at step S61′ in FIG. 25 projection data are extracted out of projection data of the same scanning position and, if only one set of projection data is extracted, it is used as projection data Dr or, if there is a plurality of sets, they are subjected to interpolation or weighted addition to obtain projection data Dr.

As a result, though the tomogram of the reconstruction plane P0.5 is obtained by only one round of image reconstruction at step S6 of FIG. 4, at step S6′ of FIG. 25 a tomogram G1 of the reconstruction plane P0.5 is image-reconstructed from the projection data obtained in the scanning position z0, and a tomogram G2 of the reconstruction plane P0.5 is image-reconstructed from the projection data obtained in the scanning position z1 as FIGS. 26(a) through 26(d) show.

Referring back to FIG. 24, at step S7, a plurality of tomograms on the same reconstruction plane are subjected to interpolation or weighted addition to obtain a single tomogram. For instance, by subjecting the tomograms G1 and G2 on the reconstruction plane P0.5 shown in FIGS. 26(a) through 26(d) to interpolation or weighted addition on a pixel-by-pixel basis, a tomogram G on the reconstruction plane P0.5 is obtained. Namely:

G=k1·G1+k2·G2

where k1 and k2 are interpolation coefficients or weighted addition coefficients, which are determined on the basis of the geometrical positions and directions of the X-ray beams passing the pixels of the tomograms to be subjected to interpolation or weighted addition. Incidentally, k1+k2=1 is supposed.

The X-ray CT apparatus of Embodiment 5 provides a picture quality improving effect and a wastefully irradiated area reducing effect to those of Embodiment 1. Furthermore, a separate tomogram is additionally obtained for each scanning position even on the same reconstruction plane.

### Embodiment 6

FIG. 27 is a flow chart of an X-ray CT imaging method pertaining to Embodiment 6.

Compared with the flow chart of the X-ray CT imaging method of Embodiment 5 shown in FIG. 24, step S7 in FIG. 24 is replaced by step S7′. Other steps are the same. Therefore, only step S7 will be described.

At step S7′, a plurality of tomograms on reconstruction planes in a prescribed z-axis direction range are subjected to interpolation or weighted addition to obtain a single tomogram.

The X-ray CT apparatus of Embodiment 6 provides a picture quality improving effect and a wastefully irradiated area reducing effect to those of Embodiment 5. Furthermore, it can control the slice thickness by appropriately setting the z-axis direction range, interpolation coefficient and weighted addition coefficient.

The X-ray CT apparatus and X-ray CT imaging method according to the present invention can be utilized picking up tomograms of a subject. It can also be utilized in medical X-ray CT apparatuses, industrial X-ray CT apparatuses or X-ray CT-PET apparatuses or X-ray CT-SPECT apparatuses combined some other apparatuses.

1. An X-ray CT apparatus comprising:
a projection data acquisition device for acquiring, while rotating an X-ray generating device and a multi-row X-ray detector opposite the X-ray generating device within an xy plane around a rotation center axis positioned between the X-ray generating device and the multi-row X-ray detector, projection data of a subject positioned in-between;
a collimator for controling the opening width of an X-ray beam irradiating the multi-row X-ray detector in a direction perpendicular to the xy plane;
a scanning table for transferring the subject in the z-axis direction;
an image reconstruction device for image-reconstructing tomograms on the basis of the projection data that have been acquired;
an image display for displaying the tomograms having undergone the image reconstruction;
a scanning condition setting device for setting various scanning conditions for acquiring the projection data; and
a controller for controlling, when performing conventional scanning (axial scanning) or cine-scanning in consecutive different scanning positions in the z-axis direction, at both scan positions, the collimator as to make the width of the X-ray beam D/2 or approximately D/2 relative to a multi-row X-ray detector width D on the rotation center axis or to make the expanding angle of the X-ray beam θ/2 or approximately θ/2 relative to a detector angle θ.

2. An X-ray CT apparatus comprising:
a projection data acquisition device for acquiring, while rotating an X-ray generating device and a multi-row X-ray detector opposite the X-ray generating device within an xy plane around a rotation center axis positioned between the X-ray generating device and the multi-row X-ray detector, projection data of a subject positioned in-between;
a collimator for controling the opening width of an X-ray beam irradiating the X-ray multi-row X-ray detector in a direction perpendicular to the xy plane;
a scanning table for transferring the subject in the z-axis direction;
an image reconstruction device for image-reconstructing tomograms on the basis of the projection data that have been acquired;
an image display for displaying the tomograms having undergone the image reconstruction;
a scanning condition setting device for setting various scanning conditions for acquiring the projection data; and
a controller for controlling, when performing conventional scanning (axial scanning) or cine-scanning in consecutive different scanning positions in the z-axis direction, the scanning table as to keep the interval between one scanning position and another scanning position at not more than D relative to a multi-row X-ray detector width D on the rotation center axis.

3. The X-ray CT apparatus according to claim 2, wherein the controller further controlling the scanning table as to keep the interval between one scanning position and another scanning position at not more than D.

4. The X-ray CT apparatus according to claim 1, comprising a projection data synthesizing device for synthesizing projection data for image reconstruction by subjecting projection data which have been acquired in different scanning positions and match the X-ray beam passing the same pixel on the reconstruction plane to interpolation or weighted addition.

5. The X-ray CT apparatus according to claim 2, comprising a projection data synthesizing device for synthesizing projection data for image reconstruction by subjecting projection data which have been acquired in different scanning positions and match the X-ray beam passing the same pixel on the reconstruction plane to interpolation or weighted addition.

6. The X-ray CT apparatus according to claim 1, comprising a projection data synthesizing device for synthesizing projection data for image reconstruction by subjecting projection data which have been acquired in different scanning positions and match the X-ray beam passing the same pixel or the vicinities of the pixel on the reconstruction plane to interpolation or weighted addition.

7. The X-ray CT apparatus according to claim 2, comprising a projection data synthesizing device for synthesizing projection data for image reconstruction by subjecting projection data which have been acquired in different scanning positions and match the X-ray beam passing the same pixel or the vicinities of the pixel on the reconstruction plane to interpolation or weighted addition.

8. The X-ray CT apparatus according to claim 1, wherein the image reconstruction device is equipped with a tomogram synthesizing device for synthesizing a new tomogram by subjecting tomograms from projection data acquired in the same scanning position to image reconstruction and subjecting tomograms having undergone image reconstruction from projection data on the same reconstruction plane in different scanning positions to interpolation or weighted addition on a pixel-by-pixel basis.

9. The X-ray CT apparatus according to claim 2, wherein the image reconstruction device is equipped with a tomogram synthesizing device for synthesizing a new tomogram by subjecting tomograms from projection data acquired in the same scanning position to image reconstruction and subjecting tomograms having undergone image reconstruction from projection data on the same reconstruction plane in different scanning positions to interpolation or weighted addition on a pixel-by-pixel basis.

10. An X-ray CT imaging method for acquiring projection data of a subject positioned between an X-ray generating device and a multi-row X-ray detector opposite the X-ray generating device while rotating the X-ray generating device and the multi-row X-ray detector within an xy plane around a rotation center axis positioned in-between,
wherein, when performing conventional scanning (axial scanning) or cine-scanning in consecutive different scanning positions in the z-axis direction orthogonal to the xy plane, at both scan positions, the width of the X-ray beam in the z-axis direction is made D/2 or approximately D/2 relative to a multi-row X-ray detector width D on the rotation center axis or the expanding angle of the X-ray beam in the z-axis direction is made θ/2 or approximately θ/2 relative to a detector angle θ.

11. An X-ray CT imaging method for acquiring projection data of a subject positioned between an X-ray generating device and a multi-row X-ray detector opposite the X-ray generating device while rotating the X-ray generating device and the multi-row X-ray detector within an xy plane around a rotation center axis positioned in-between,
wherein, when performing conventional scanning (axial scanning) or cine-scanning in consecutive different scanning positions in the z-axis direction orthogonal to the xy plane, the interval between one scanning position and another scanning position is kept at not more than D.

12. The X-ray CT imaging method according to claim 11, wherein the interval between one scanning position and another scanning position is kept at not more than D.

13. The X-ray CT imaging method according to claim 10, wherein tomograms are subjected to image reconstruction on the basis of projection data obtained by subjecting projection data which have been acquired in different scanning positions and match the X-ray beam passing the same pixel on the reconstruction plane to interpolation or weighted addition.

14. The X-ray CT imaging method according to claim 11, wherein tomograms are subjected to image reconstruction on the basis of projection data obtained by subjecting projection data which have been acquired in different scanning positions and match the X-ray beam passing the same pixel on the reconstruction plane to interpolation or weighted addition.

15. The X-ray CT imaging method according to claim 10, wherein projection data for image reconstruction are synthesized by subjecting projection data which have been acquired in different scanning positions and match the X-ray beam passing the same pixel or the vicinities of the pixel on the reconstruction plane to interpolation or weighted addition.

16. The X-ray CT imaging method according to any of claim 11, wherein projection data for image reconstruction are synthesized by subjecting projection data which have been acquired in different scanning positions and match the X-ray beam passing the same pixel or the vicinities of the pixel on the reconstruction plane to interpolation or weighted addition.

17. The X-ray CT imaging method according to claim 10, further comprising the steps for image reconstructing tomograms from projection data acquired in the same scanning position and synthesizing a new tomogram by subjecting tomograms having undergone image reconstruction from projection data on the same reconstruction plane in different scanning positions to interpolation or weighted addition on a pixel-by-pixel basis.

18. The X-ray CT imaging method according to claim 11, further comprising the steps for image reconstructing tomograms from projection data acquired in the same scanning position and synthesizing a new tomogram by subjecting tomograms having undergone image reconstruction from projection data on the same reconstruction plane in different scanning positions to interpolation or weighted addition on a pixel-by-pixel basis.
